(19) United States
(12) Patent Application Publication
(10) Pub. No.: US 2023/0263930 A1
(43) Pub. Date: Aug. 24, 2023

(54) STERILIZATION DEVICE AND STERILIZATION SYSTEM

(71) Applicant: KALTECH CORPORATION, Osaka-shi, Osaka (JP)

(72) Inventor: Junichi SOMEI, Osaka (JP)

(21) Appl. No.: 18/040,244

(22) PCT Filed: Nov. 10, 2020
(86) PCT No.: PCT/JP2020/041827

§ 371 Date: Feb. 1, 2023

(30) Foreign Application Priority Data
Aug. 4, 2020 (JP) 2020-132235

(51) Int. Cl. A61L 9/20

(52) U.S. Cl. CPC A61L 9/205; A61L 2209/134; A61L 2209/14

(57) ABSTRACT

Provided herein is a sterilization device 100 that includes, a cylindrical case 110 having an air outlet 112 on the top and an air inlet 111 on the side, a fan 140 arranged below the air outlet 112 in the case 110 for sending air to the air outlet 112, and a filter 121 arranged below the fan 140 in the case 110.

### TECHNICAL FIELD

The present invention relates to a technology of a sterilization device for sterilization and deodorization of air with the use of a photocatalyst.

### BACKGROUND ART

Photocatalytic devices for deodorizing and disinfecting air have been known. For example, According to Japanese Patent Laying-Open No. 2019-5094 (Patent Document 1), a compact air purifying device comprises a tubular casing having an air inlet and an air outlet, and an air purifying unit housed in the casing. The air cleaning unit includes an intake fan for introducing air into the casing from the intake port, a cleaning section arranged between the intake fan and the exhaust port, and at least one light emitting section for irradiating the cleaning section with light. The intake fan, the cleaning section, and the light emitting section are provided along the axial direction of the casing. The cleaning section includes a plate-like filter through which air passes, and a masking section that is arranged on the periphery of the filter and blocks the passage of air. A space within the casing is divided along a direction perpendicular to the axial direction of the casing. The filter comprises a porous plate and a photocatalyst carried on the porous plate and purifies the air passing through the filter by light irradiation from the light emitting part.

Patent Document 1: JP2019-5094

### DISCLOSURE OF INVENTION

### Technical Problem

An object of the present invention is to provide a sterilization device for output sterilized air from the end of a cylindrical case.

### Solution to Problem

According to a certain aspect of the present invention, there is provided a sterilization device that include, a cylindrical case having an air outlet on the top and an air inlet on the side, a fan arranged below the air outlet in the case for sending air to the air outlet, and a filter arranged below the fan in the case.

### Advantageous Effects of Invention

It can thus be seen that the present invention has provided a sterilization device for output sterilized air from the end of a cylindrical case. As a result, when hanging the device from the neck, it is possible to supply sterile air to the vicinity of the user's face.

### BRIEF DESCRIPTION OF THE DRAWINGS

FIG. 1 is a front perspective view showing the sterilization device according to First Embodiment.

FIG. 2 is a rear perspective view showing the sterilization device according to First Embodiment.

FIG. 3 is a plan view showing the sterilization device according to First Embodiment.

FIG. 4 is a cross sectional view at A-A of FIG. 3.

FIG. 5 is a cross sectional view at B-B of FIG. 3.

FIG. 6 is a front perspective view showing the sterilization system according to First Embodiment.

FIG. 7 is a front view showing the sterilization system according to First Embodiment.

FIG. 8 is an assembly exploded view of the sterilization device according to First Embodiment.

FIG. 9 is a front perspective view showing the cover according to First Embodiment.

FIG. 10 is an assembly exploded view of the sterilization system according to First Embodiment.

FIG. 11 is an enlarged view showing the lower end of neck frame according to First Embodiment.

FIG. 12 is a front cross sectional view showing the same according to First Embodiment.

FIG. 13 is a cross sectional view at C-C of FIG. 9.

FIG. 14 is a front perspective view showing the sterilization system according to First Embodiment.

FIG. 15 is a view showing the sterilization system according to First Embodiment.

FIG. 16 is an upper perspective view of a charging device according to First Embodiment.

FIG. 17 is a cross sectional view at D-D of FIG. 15.

FIG. 18 is a right side cross-sectional view of the charging device at the position of the charging terminal according to First Embodiment.

FIG. 19 is a front perspective view showing the sterilization system with the sterilization device installed in the charging station facing upside down according to First Embodiment.

FIG. 20 is a diagram showing the bridge circuit for charging according to First Embodiment.

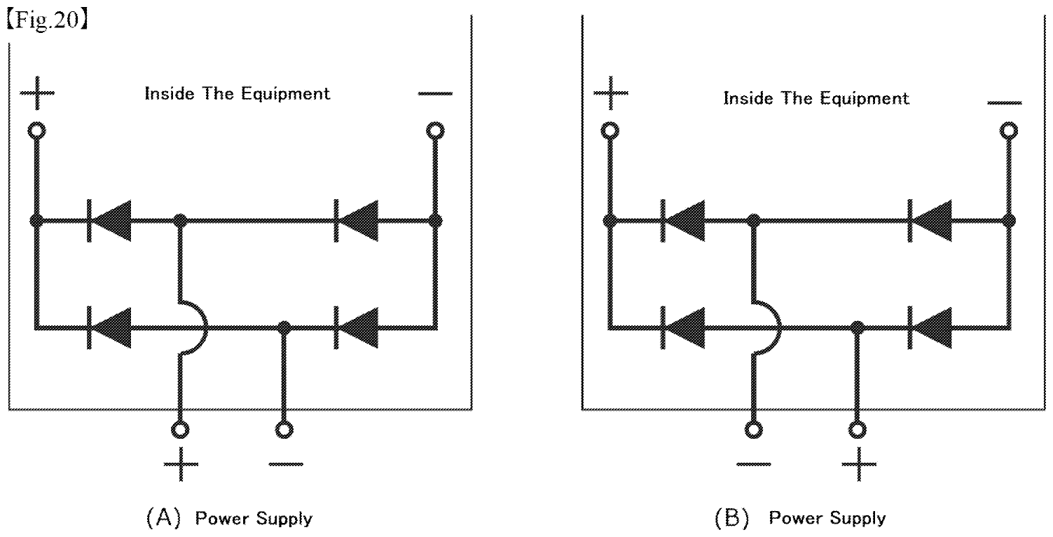

### DESCRIPTION OF THE PREFERRED EMBODIMENTS

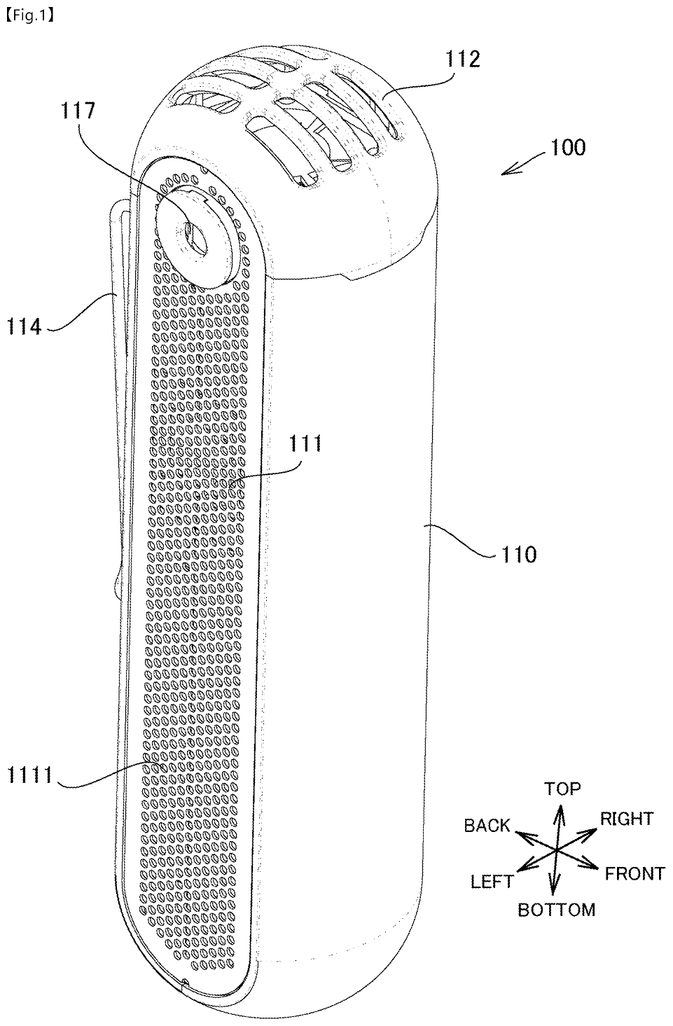

The following describes embodiments of the present invention with reference to the accompanying drawings. In the following descriptions, like elements are given like reference numerals. Such like elements will be referred to by the same names, and have the same functions. Accordingly, detailed descriptions of such elements will not be repeated. In the following descriptions of sterilization device 100, for the sake of explanation, the terms used to refer to directions, for example, front, back, left, right, top, and bottom, are relative to the position of the state in which the air outlet is directed upward and the clip is directed backward, as shown in FIG. 1.

### First Embodiment

### Configuration of the Main Body of Sterilization Device 100

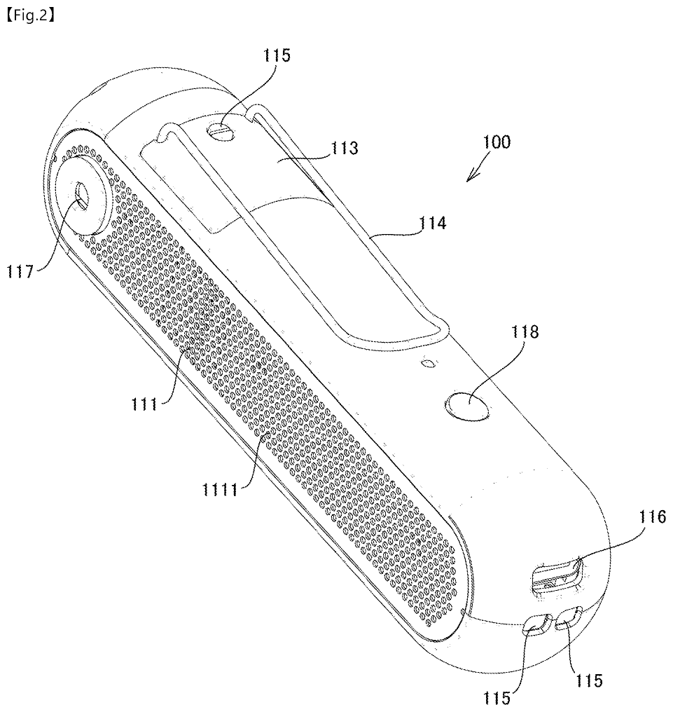

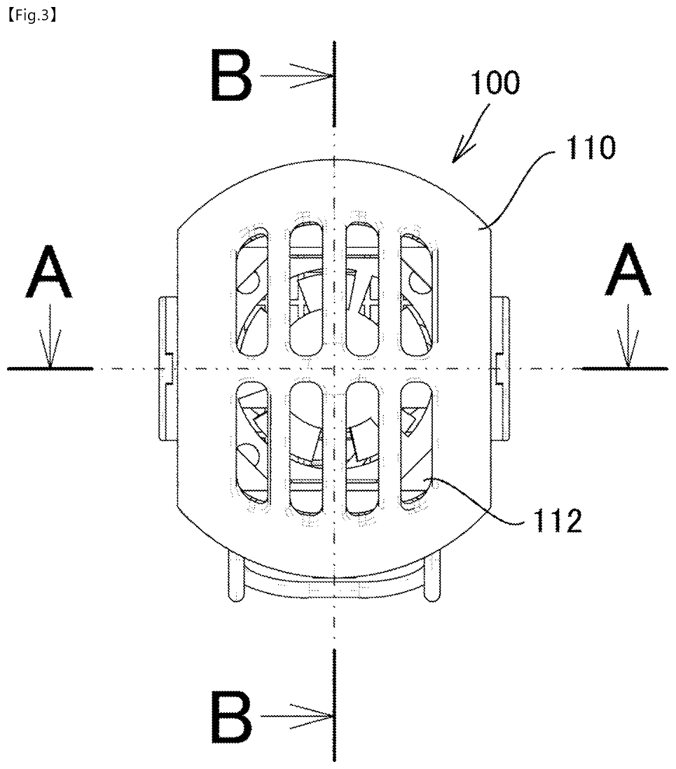

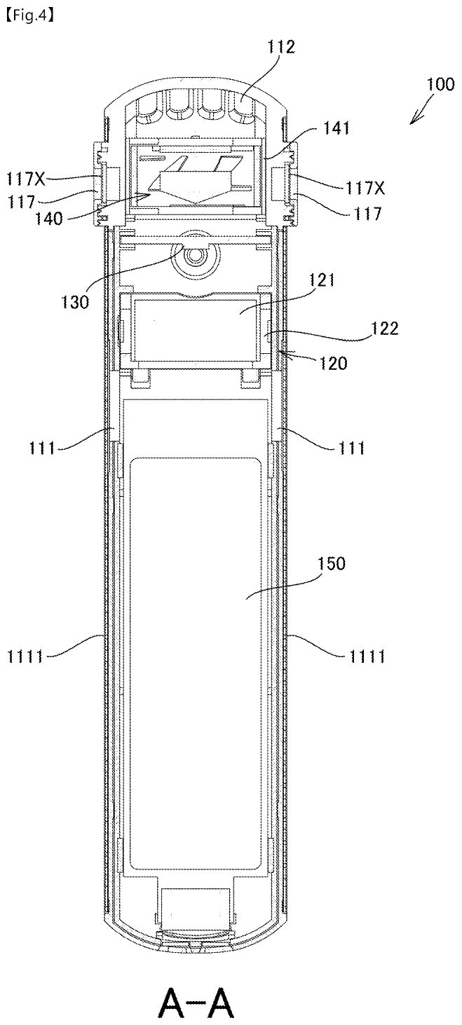

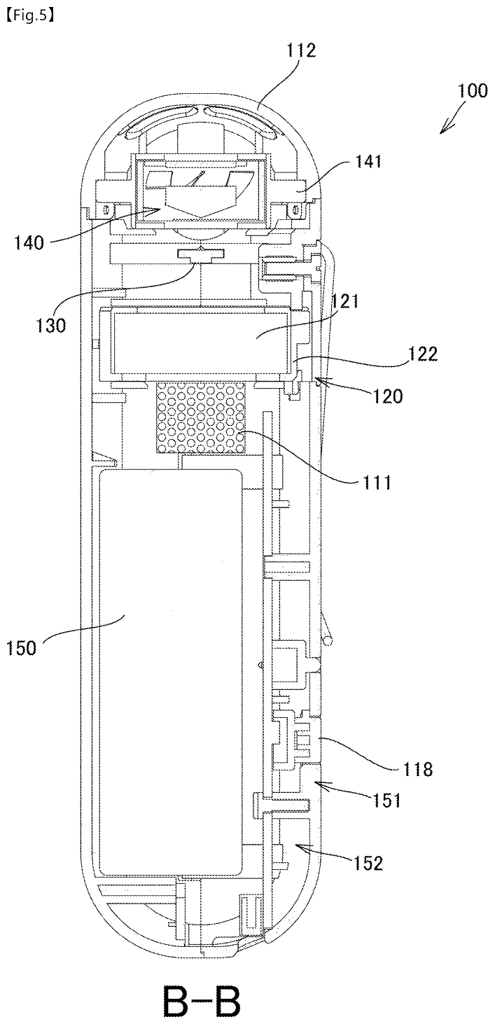

First, the structure of the main body of the sterilization device 100 according to this embodiment will be described. Referring to FIGS. 1 to 5, the sterilization device 100 according to the present embodiment includes, as main components, a photocatalyst unit 120, an LED light 130, a blow fan 140, and a case 110 for housing them.

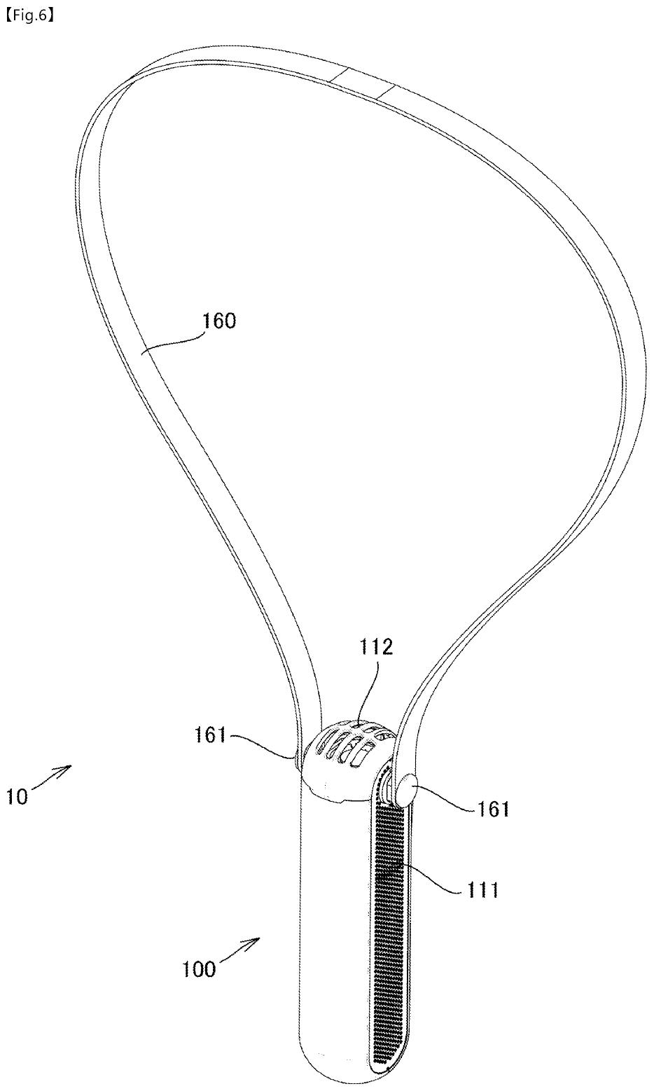

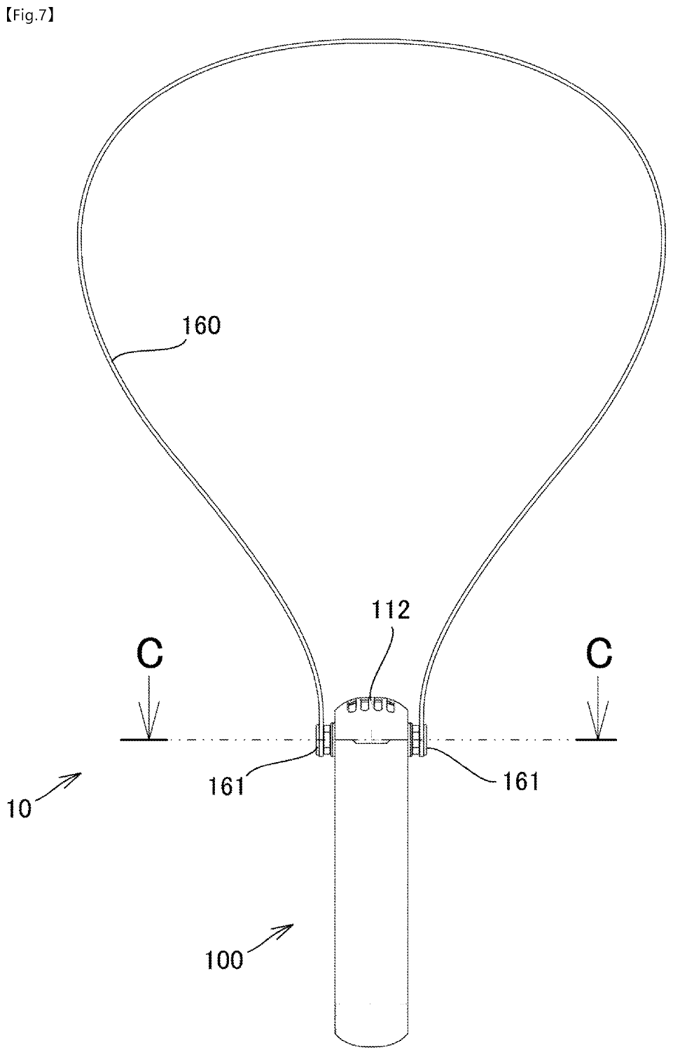

As shown in FIGS. 6 and 7, in the present embodiment, a neck frame 160 is attached to the top of case 110. The user can hold the sterilization device 100 directly under the face by hanging the neck frame 160 around the neck. In this state, the sterilization device 100 blows out sterilized air from its upper part upward, that is, near the user's face. The main body of the sterilization device 100, the neck frame 160, the charging device 180, and the like are collectively referred to as the sterilization system 10.

Moreover, in the present embodiment, a clip 114 is attached to the rear surface of the sterilization device 100.

The user can hold the sterilization device 100 directly under the face by inserting clothing such as a shirt, a bag, a belt, or the like between the clip 114 and the back surface of the case 110 of the sterilization device 100. In this state, the sterilization device 100 blows the sterilized air upward from the blowout port 112 at the top of the device.

Returning to FIG. 1 to FIG. 5, inlet ports 111 and 111 are formed in case 110 according to the present embodiment. More specifically, a large number of punched holes are formed in the left and right side surfaces 1111 and 1111. The inlet ports 111 and 111 connected to the internal space of the case 110 are formed in the upper middle portions of the left and right side surfaces 1111 and 1111. A rechargeable battery 150 is arranged in the lower part inside the case 110. A blow fan 140 and a photocatalyst unit 120 are arranged in the upper part inside the case 110. The inlet ports 111, 111 are formed at positions avoiding the rechargeable battery 150, the photocatalyst unit 120, and the like.

Inside the case 110, from below, the rechargeable battery 150, the photocatalyst unit 120, the LED light 130, and the blow fan 140 are arranged in this order. In this embodiment, inlet ports 111 and 111 are formed between the rechargeable battery 150 and the photocatalyst unit 120 in side view.

A control board 151 is arranged on the back side of the rechargeable battery 150. An ON/OFF switch 118 is provided near the control board 151. The switch 118 protrudes outside from the back surface of the case 110. The control board 151 is also provided with a bridge circuit 152 for charging, which will be described later.

The bottom surface of case 110 is formed in a curved shape. Charging terminals 115 and 115 and a USB terminal 116 are provided on the bottom surface of the case 110. These power supply terminals are connected to the control board 151.

In this embodiment, the photocatalyst unit 120 includes a photocatalyst filter 121 and a holder 122 holding the photocatalyst filter 121 inside. The photocatalyst filter 121 has a large number of openings of the vertical direction of the case 110. For example, the photocatalyst filter 121 is formed like a grid.

Here, the photocatalyst according to the present embodiment decomposes the oxygen and moisture in the air adsorbed on the photocatalyst's surface by a photoreaction, to generate highly reactive superoxide anions (O2—) and OH radicals (.OH). Superoxide anions (O2—) and OH radicals (.OH) decompose odorous components, remove various bacteria, and decompose ethylene gas to suppress deterioration of freshness of foods. Examples of photocatalysts include zinc oxide (ZnO), cadmium sulfide (CdS), tungsten trioxide (WO3), and titanium dioxide (TiO2), but photocatalysts are not limited to these.

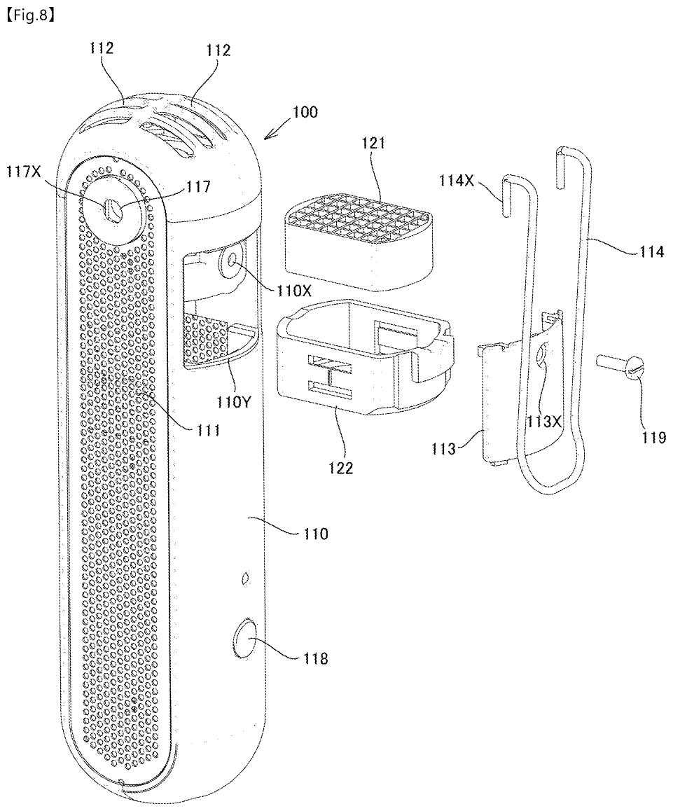

Then, as shown in FIG. 8, the photocatalyst unit 120 is configured to be detachable from the case 110. More specifically, an opening 110Y is formed in a portion of the rear surface of the case 110, that is, the rear portion of the photocatalyst unit 120. The opening 110Y is covered with a cover 113 that is detachable from the case 110. The cover 113 is attached to the rear surface of the case 110 with screws 119 or the like. The screw 119 passes through the screw hole 113X of the cover 113 and is screwed into the screw hole 110X of the case 110.

With this configuration, the user can remove the cover 113 from the case 110 by removing the screw 119. Then, the user can periodically take out the photocatalyst unit 120 to the outside through the opening 110Y. The user can easily wash the photocatalyst filter 121 with water or the like while the photocatalyst filter 121 is attached to the holder 122. At that time, the user does not need to touch the photocatalyst filter 121 directly.

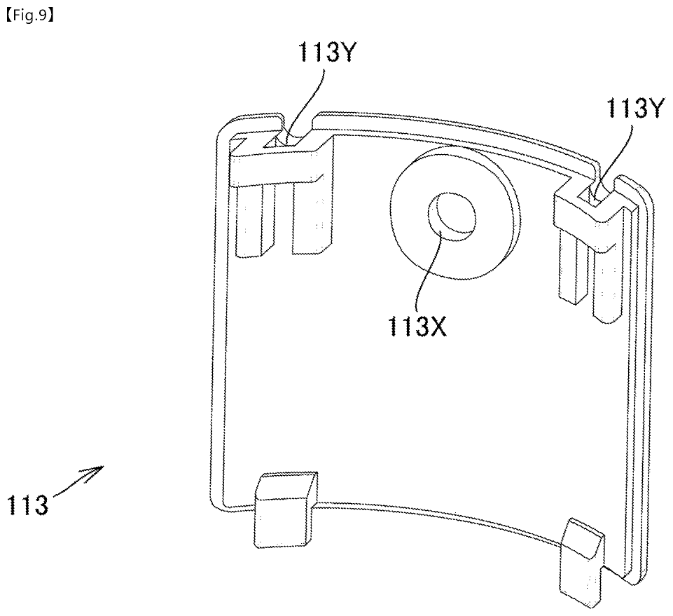

In particular, in this embodiment, the upper end of clip 114 described above is attached to cover 113. More specifically, as shown in FIG. 9, two holes 113X and 113X are formed on the front side of the upper portion of the cover 113, that is, on the inner side of the sterilization device 100. Thereby, the downward bent portion 114X of the upper end of the clip 114 is fixed to the cover 113 by inserting into the two holes 113X, 113X. Thus, in the sterilization device 100 of the present embodiment, the clip 114 is detachably attached to the cover 113. Therefore, the clip 114 can be attached or detached from the sterilization device 100 with the cover 113 removed from the device, depending on the user's preference.

Returning to FIGS. 1 to 5, above the photocatalyst unit 120, the LED light 130 is attached for irradiating the photocatalyst filter 121 with light. Power is supplied to the LED lights 130 from the control board 151.

The blow fan 140 is attached above the LED light 130. In this embodiment, an axial fan is used. Power is supplied from the control board 151 to the blow fan 140. In the present embodiment, blow fan 140 is configured to blow air toward outlet 112 above. That is, the area in which the photocatalyst unit 120 and the LED light 130 are arranged becomes negative pressure. The blowout port 112 is provided near the blow fan 140. The air sucked by the inlet port 111 can be smoothly flowed.

In particular, blow fan 140 is held by case 110 in this embodiment. An elastic member 141 such as rubber is sandwiched between the outer circumference of the blow fan 140 and the inner surface of the case 110. As a result, the vibration of the blow-out fan is less likely to be transmitted to the case 110. In addition, the elastic member 141 is provided on the side surface of the unit of the blow fan 140 for one round. A gap is not formed between the side surface of the blow fan 140 unit and the case 110. Air does not escape from the gap between the unit of the blow fan 140 and the inner surface of the case 110.

### Neck Frame 160

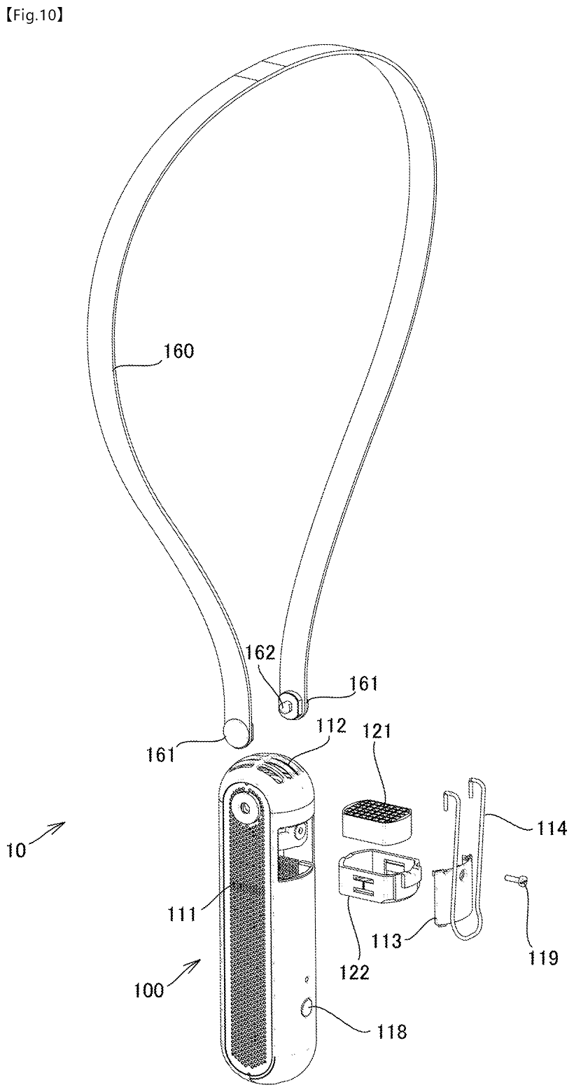

Next, the configuration of the neck hanging of the sterilization device 100 according to the present embodiment will be described. As shown in FIGS. 6 and 10, a neck frame 160 is detachably attached to the sterilization device 100.

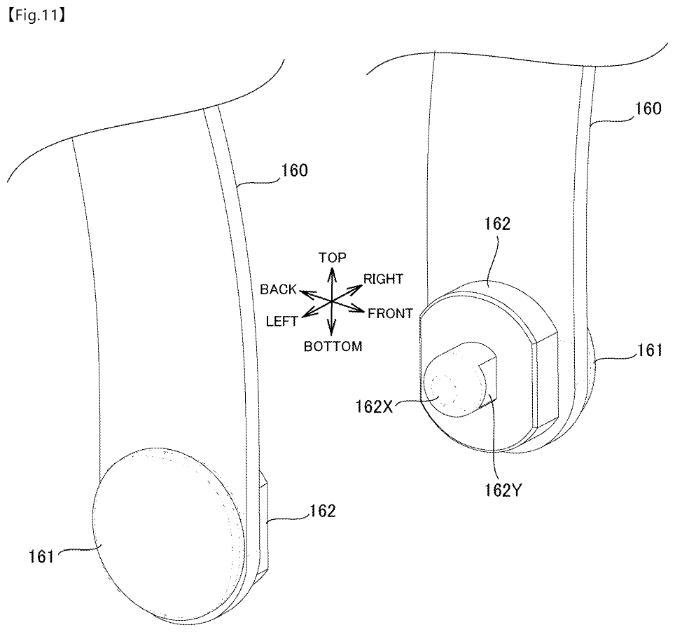

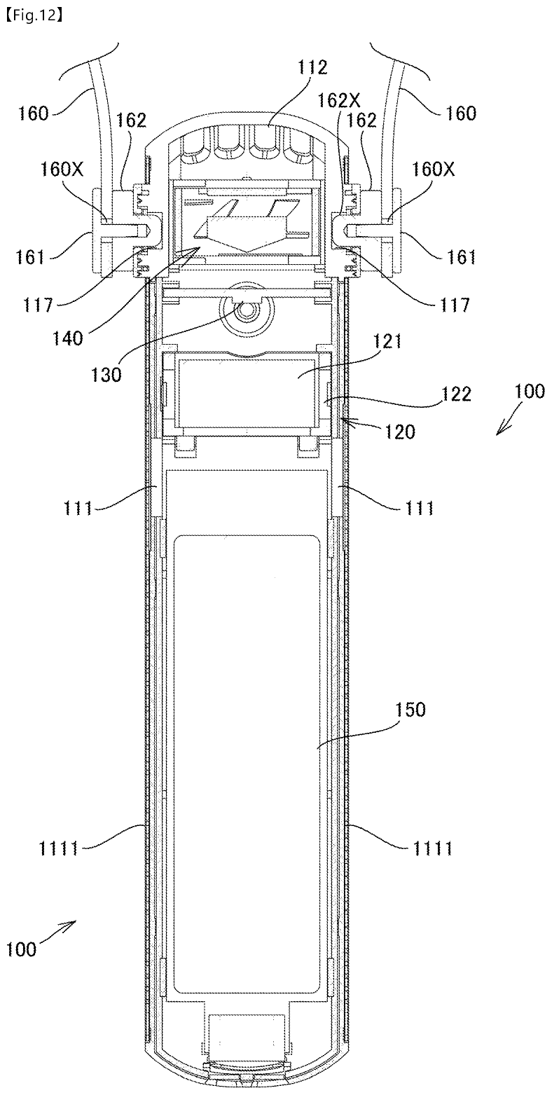

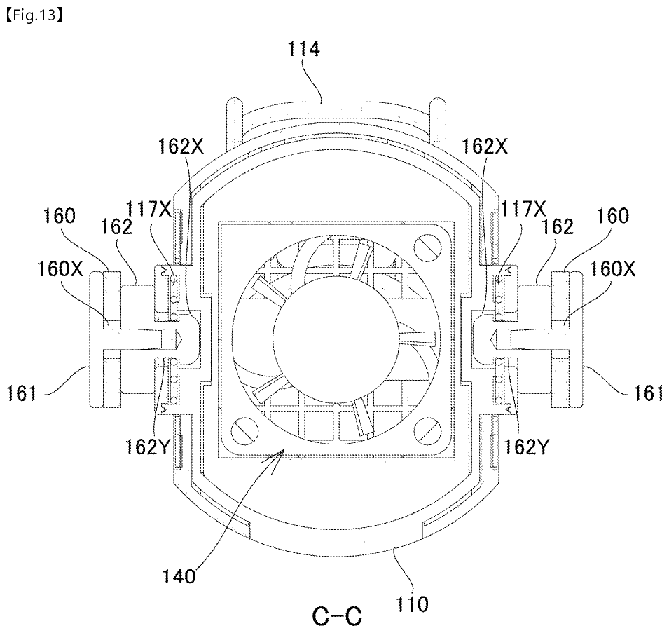

In the following, for the sake of explanation, the terms used to refer to directions, for example, front, back, left, right, top, and bottom, are relative to the position of the state in which the sterilization device 100 and the neck frame 160 stand vertically, as shown in FIGS. 6 to 13.

The neck frame 160 has inward projections 162X formed at both left and right lower ends. More specifically, holes 160X, 160X are formed in the lower end of the body of the neck frame 160. Locking members 161, 161 are respectively inserted into the holes 160X, 160X from the outside direction of the neck frame 160 and the sterilization device 100. Protruding members 162 and 162 are fitted to the tips of the locking members 161 and 161 from the inner side of the neck frame 160 and the sterilization device 100. The neck frame 160 is strongly sandwiched between the locking members 161 and 161 and the projecting members 162 and 162. The postures of the locking members 161 and 161 and the projecting members 162 and 162 with respect to the neck frame 160 are fixed.

Projections 162X and 162X are respectively formed on the projection members 162 and 162 toward the inside of the neck frame 160 and the sterilization device 100. Front surfaces 162Y and 162Y and rear surfaces of the central portions in the left-right direction of the protrusions 162X and 162X are cut. That is, the left and right central portions of the protrusions 162X, 162X are formed such that the distance between the upper surface and the lower surface is long, and the distance between the front surfaces 162Y, 162Y and the rear surface is short.

On the other hand, with respect to the case 110 of the sterilization device 100, mounting holes 117, 117 are formed in the upper portions of the left and right side surfaces 1111, 1111. Elastic members 117X and 117X, on the front and rear surfaces of the inner surfaces of the mounting holes 117 and 117, protrude toward the center of the mounting holes 117 and 117. Because of this configuration, when the neck frame 160 is laid down horizontally, the elastic members 117X and 117X can be pushed outward by the central portions of the protrusions 162X and 162X. The protrusions 162X, 162X can be inserted into the mounting holes 117,117. More specifically, the protrusions 162X and 162X can be inserted into the mounting holes 117 and 117 by pushing the elastic members 117X and 117X forward and backward with the upper and lower surfaces of the central portions of the protrusions 162X and 162X. When the neck frame 160 is raised again, the central front surfaces 162Y and 162Y and the rear surfaces of the projections 162X and 162X are no longer able to push the elastic members 117X and 117X away. The tip portions of the projections 162X, 162X are caught on the elastic members 117X, 117X. The protrusions 162X, 162X cannot be removed from the mounting holes 117, 117.

By horizontally laying down the neck frame 160 again, the upper and lower surfaces of the central portions of the protrusions 162X and 162X push the elastic members 117X and 117X away. This allows the protrusions 162X, 162X to be pulled out from the mounting holes 117, 117.

That is, in the present embodiment, when the user wears the sterilization device 100 around the neck, the sterilization device 100 rotates around the mounting holes 117 and 117, and the outlet 112 faces upward. It becomes difficult for the sterilization device 100 to come off from the neck frame 160.

### Charging Mechanism

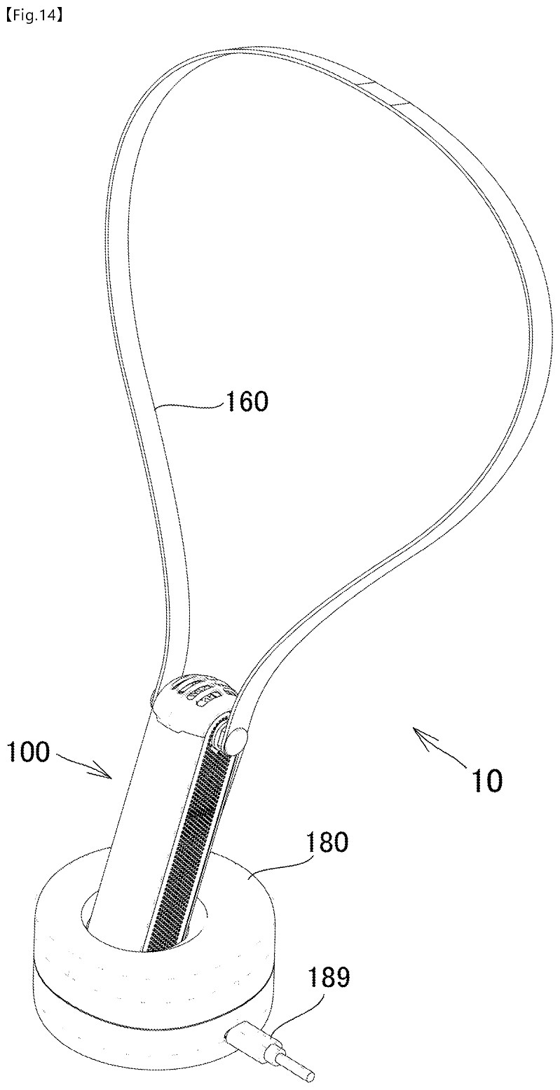

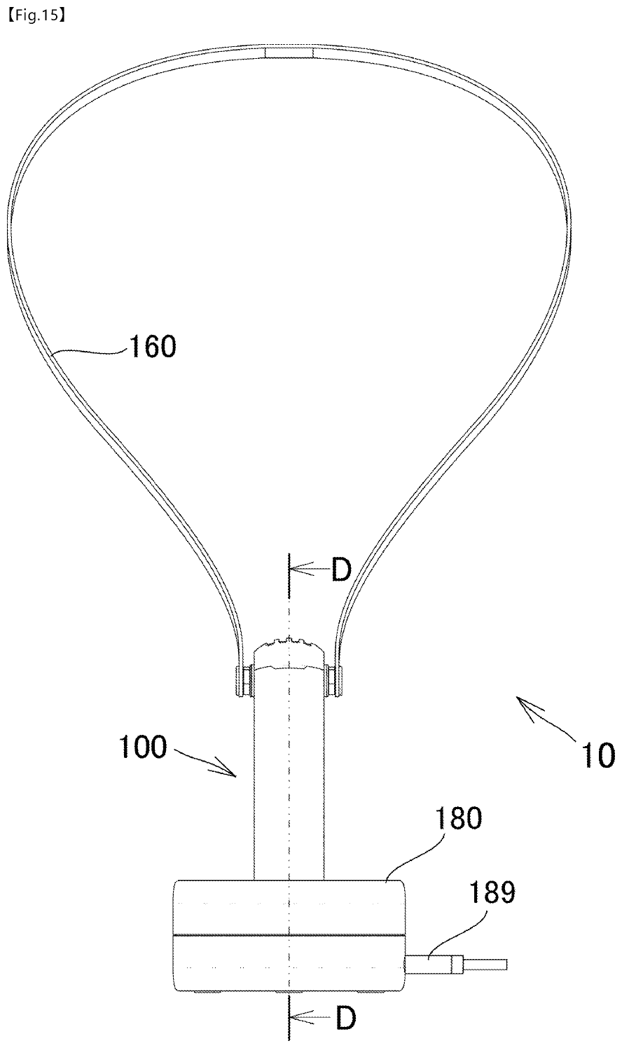

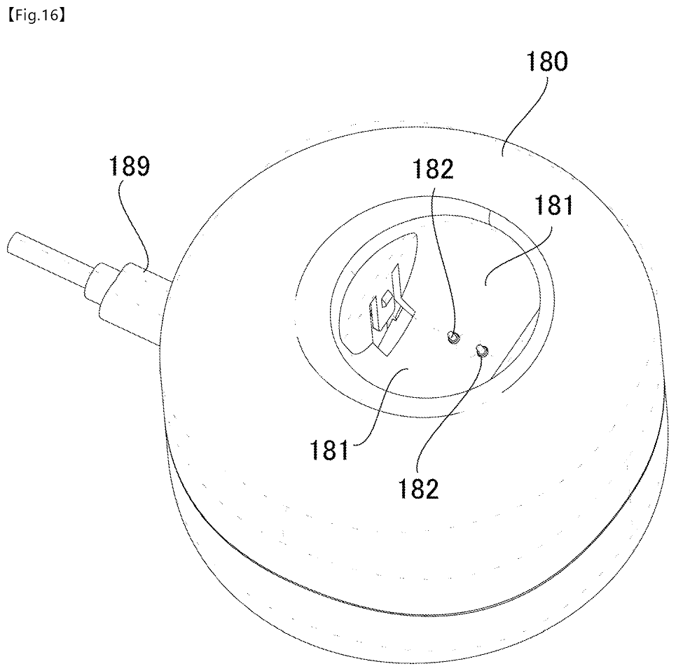

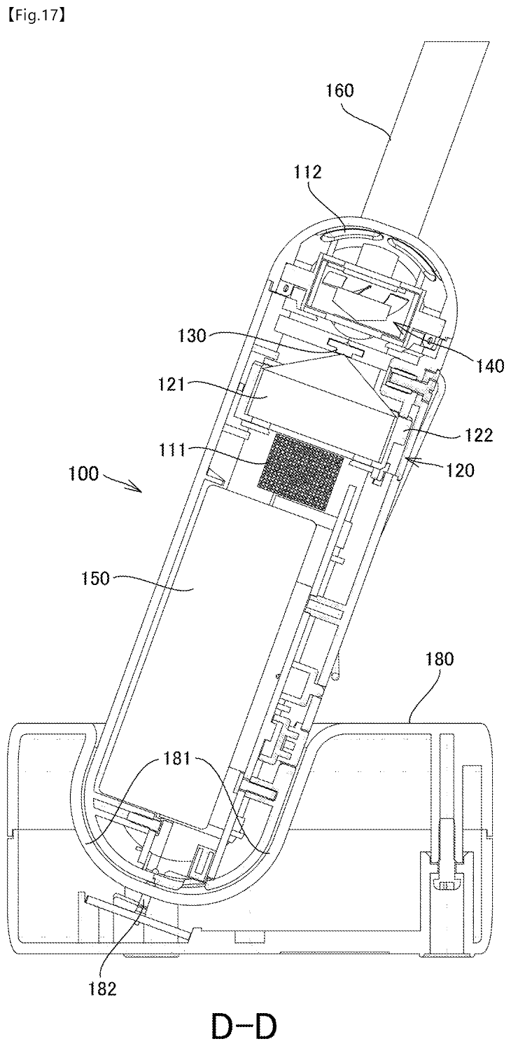

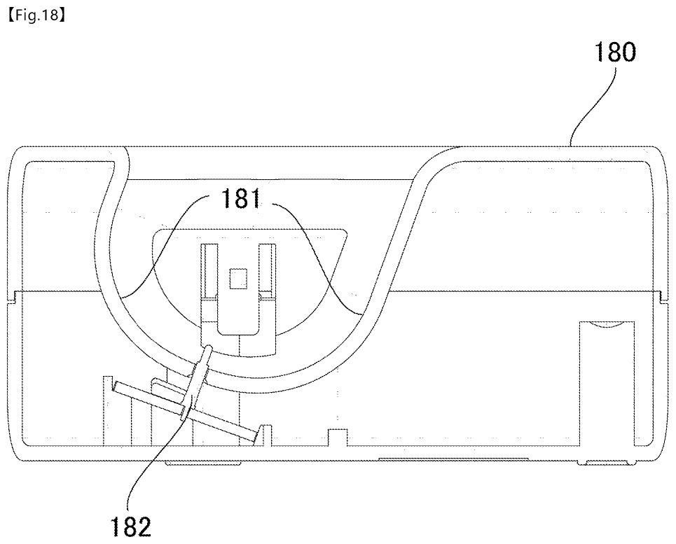

FIGS. 14 to 18, the sterilization device 100 according to this embodiment is charged by being placed on a stand-type charging device 180. The charging device 180 obtains, via a USB cable 189, power from a power outlet, a USB connector of a personal computer, or the like.

The charging device 180 according to the present embodiment is formed in a columnar shape with a low height. A concave portion 181 is formed in the central portion of it in plan view. By inserting the lower end of the sterilization device 100 into the recess 181, the sterilization device 100 is held in an upright state. In this embodiment, the sterilization device 100 is fixed in a state of being tilted toward the back of the sterilization device 100 according to the shape of the recess 181.

Two charging terminals 182, 182, that is, a positive terminal 182 for charging and a negative terminal 182 for charging, protrude from the bottom surface of the recess 181. Two charging terminals 182, 182 are provided at specific positions. The two charging terminals 182, 182 are in contact with the charging terminals 115, 115 on the bottom surface of the sterilization device 100 while the sterilization device 100 is fixed in an oblique state according to the shape of the recess 181. The rechargeable battery 150 of the sterilization device 100 is charged by connecting the charging terminals 182 and 182 of the charging device 180 to the charging terminals 115 and 115 on the bottom of the sterilization device 100.

The charging terminals 182, 182 of the concave portion 181 are slidable in the vertical direction. Charging terminal 182 is biased upward. As a result, when the lower end of the sterilization device 100 is inserted into the recess 181, the charging terminals 182 and 182 are pushed downward by the bottom surface of the sterilization device 100. In this state, the sterilization device 100 shifts to a tilted posture by the recess 181. When the sterilization device 100 stabilizes in the tilted state, the charging terminals 182, 182 biased upward are automatically connected to the charging terminals 115, 115 of the sterilization device 100.

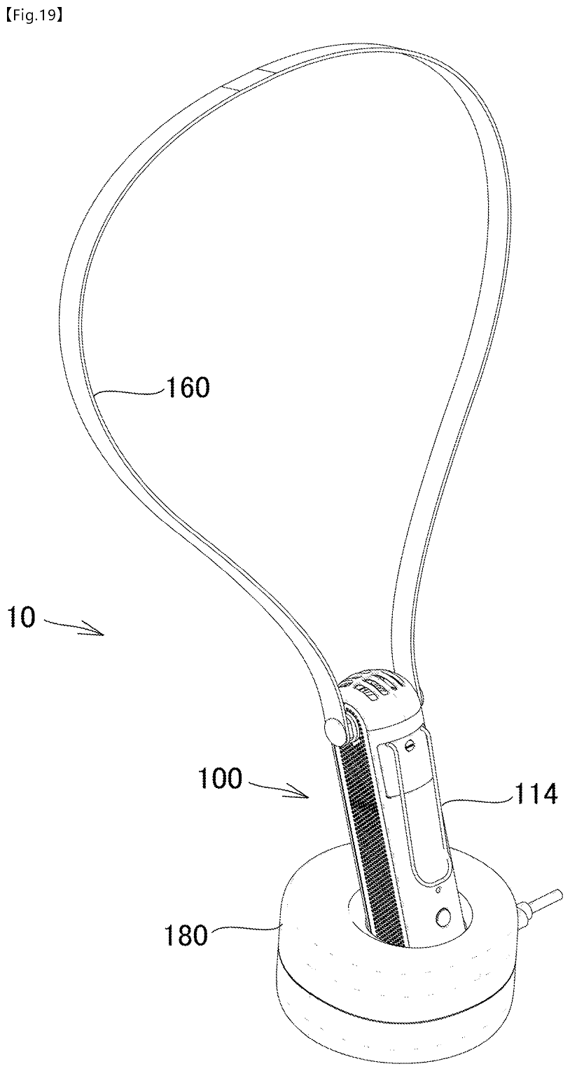

In addition, in the present embodiment, the shape of the lower end portion of the case 110 of the sterilization device 100 is substantially line-symmetrical in the front-rear direction. The shape of the lower end portion of the case 110 of the sterilization device 100 is also substantially line-symmetrical in the horizontal direction. More precisely, the shape of the lower end portion of the case 110 of the sterilization device 100 is symmetrical about a vertical axis passing through the front-rear center and the left-right center. Therefore, as shown in FIG. 19, the sterilization device 100 is configured such that its lower end fits into the recess 181 of the charging device 180 even when it is turned upside down in the front-rear direction.

Also, the positions of the charging terminals 115, 115 at the lower end of the case 110 of the sterilization device 100 are formed substantially line-symmetrically in the front-rear direction. The positions of the charging terminals 115, 115 are also substantially symmetrical in the left-right direction. More precisely, the positions of the charging terminals 115, 115 at the lower end of the case 110 of the sterilization device 100 are symmetrical about a vertical axis passing through the center in the front-rear direction and the center in the left-right direction. Therefore, as shown in FIG. 19, even if the sterilization device 100 is installed in the charging device 180 with the front and back reversed, the charging terminals 182, 182 of the charging device 180 are connected to the charging terminals 115, 115 of the sterilization device 100.

Then, as shown in FIG. 19, the wiring for charging the sterilization device 100 has a bridge circuit using diodes. With this configuration, no matter which side faces the front the sterilization device 100 is fitted into the charging device 180, that is, whichever charging terminal 115, 115 of the sterilization device 100 the positive and negative sides of the charging terminals 182, 182 are connected, the sterilization device 100 can be charged.

### Second Embodiment

In the foregoing embodiment, the neck frame 160 is made of an elastic member such as resin. However, neck frame 160 may be formed by a string, chain, or the like.

### Third Embodiment

In the foregoing embodiments, the sterilization device 100 has one photocatalyst filter 121 and one LED light 130. However, the sterilization device 100 may include multiple photocatalyst filters or multiple LED lights.

### Forth Embodiment

Also, the light for activating the photocatalyst is not limited to the LED light 130. Other types may be used.

### Fifth Embodiment

Also, the filter for disinfecting and deodorizing the sucked air is not limited to the photocatalyst filter 121. Instead of the photocatalyst filter 121, or in addition to the photocatalyst filter, the sterilization device 100 may have other types of air cleaning filters. The position of the filter is preferably above the inlet port 111 and below the blow fan 140. That is, the filter is preferably arranged downstream of the inlet port 111 and upstream of the blow fan 140.

### Review

The foregoing embodiments provide a sterilization device that includes, a cylindrical case having an air outlet on the top and an air inlet on the side, a fan arranged below the air outlet in the case for sending air to the air outlet, and a filter arranged below the fan in the case.

Preferably, the filter is a photocatalyst filter having a plurality of openings in the wind direction of the fan. The sterilization device further includes a light arranged below the fan for irradiating the photocatalyst filter with light.

Preferably, the sterilization device further includes an elastic member arranged around the fan for supporting the fan with respect to the case and for closing between the fan and the case.

Preferably, the case has an opening in the side of the filter. The sterilization device further includes a detachable cover for closing the opening. The filter can be removed from the case by sliding it through the opening and out of the case, with the cover removed.

Preferably, the sterilization device further includes a clip attached to the back of the case for holding the sterilization device. The end of the clip is detachably attached to the cover.

Preferably, the sterilization device further includes a holder for holding the side of the filter. The filter, together with the holder, can be removed from the case by sliding it through the opening to the outside of the case.

Preferably, the sterilization device further includes a rechargeable battery. The lower end of the case has two charging terminals. The rechargeable battery can be charged regardless of which of the two charging terminals the positive terminal and the negative terminal of the charging device are connected to, by using a bridge circuit using a plurality of diodes between the charging terminal and the rechargeable battery.

Preferably, the foregoing embodiments provide a sterilization system that includes, the above sterilization device and a charging device having a recess into which the lower portion of the sterilization device can be inserted for holding the sterilization device in an upright state, and the positive terminal and the negative terminal provided on the bottom surface of the recess.

Preferably, the foregoing embodiments provide a sterilization system that includes, the above sterilization device and a detachable neck frame on top of the sterilization device.

Preferably, the upper portions of both side surfaces of the case have holes connectable to the lower ends of the neck frames. Side surfaces of each of the holes have a member biased toward the inside of the hole. Each of the lower ends of the neck frame has a protrusion that inserts into the hole. The side surface of the protrusion has a concave portion at a position facing the member.

The embodiments disclosed herein are to be considered in all aspects only as illustrative and not restrictive. The scope of the present invention is to be determined by the scope of the appended claims, not by the foregoing descriptions, and the invention is intended to cover all modifications falling within the equivalent meaning and scope of the claims set forth below.

### REFERENCE SIGNS LIST

10 sterilization system
100 sterilization device
110 case
110X screw hole
111 inlet port
1111 side surface
112 blowout port
113 cover
113X screw hole
114 clip
114X bent portion
115 charging terminal
116 USB terminal
117 mounting hole
117X elastic member
118 switch
119 screw
120 photocatalyst unit
121 photocatalyst filter
122 holder
130 LED light
140 blow fan
141 elastic member
150 rechargeable battery
151 charging terminal
152 bridge circuit
160 neck frame
160X hole
161 locking member
162 front surface
162X protrusion
162Y front surface
180 charging device
181 recess
182 charging terminal
189 USB cable

1. A sterilization device, comprising:
a cylindrical case having an air outlet on the top and an air inlet on the side;
a fan arranged below the air outlet in the case for sending air to the air outlet; and
a filter arranged below the fan in the case.

2. The sterilization device according to claim 1, wherein
the filter is a photocatalyst filter having a plurality of openings in the wind direction of the fan, and
the sterilization device further comprises a light arranged below the fan for irradiating the photocatalyst filter.

3. The sterilization device according to claim 1, further comprising:
an elastic member arranged around the fan for supporting the fan with respect to the case and for closing between the fan and the case.

4. The sterilization device according to claim 1, wherein
the case has an opening in the side of the filter,
the sterilization device further comprises a detachable cover for closing the opening, and
the filter can be removed from the case by sliding it through the opening and out of the case, with the cover removed.

5. The sterilization device according to claim 4, further comprising:
a clip attached to the back of the case for holding the sterilization device, wherein,
the end of the clip is detachably attached to the cover.

6. The sterilization device according to claim 4, further comprising:
a holder for holding the side of the filter, wherein
the filter, together with the holder, can be removed from the case by sliding it through the opening to the outside of the case.

7. The sterilization device according to claim 1, further comprising:
a rechargeable battery, wherein
the lower end of the case has two charging terminals, and
the rechargeable battery can be charged regardless of which of the two charging terminals the positive terminal and the negative terminal of the charging device are connected to, by using a bridge circuit having a plurality of diodes between the charging terminal and the rechargeable battery.

8. The sterilization system, comprising:
the sterilization device according to claim 1; and
a charging device having
a recess into which the lower portion of the sterilization device can be inserted for holding the sterilization device in a slanted state, and
the positive terminal and the negative terminal provided on the bottom surface of the recess.

9. The sterilization system, comprising:
the sterilization device according to claim 1; and
a neck frame detachable to the top of the sterilization device.

10. The sterilization system, according to claim 8, wherein
the upper portions of both side surfaces of the case have holes connectable to the lower ends of the neck frames, side surfaces of each of the holes have a member biased toward the inside of the hole, each of the lower ends of the neck frame has a protrusion for inserting into the hole, and the side surface of the protrusion has a concave portion at a position facing the member.
